(19) United States
(12) Patent Application Publication
(10) Pub. No.: US 2002/0098186 A1
(43) Pub. Date: Jul. 25, 2002

(54) Use of endothelin inhibitors for treatment or prevention of fibrotic disorders

(76) Inventors: Detlef Schuppan, Erlangen (DE); Manfred Raschack, Weisenheim (DE)

Correspondence Address: Herbert B. Keil, KEIL & WEINKAUF, 1101 Connecticut Avenue, N.W., Washington, DC, 20036, US

(21) Appl. No.: 10/043,317

(22) Filed: Jan. 14, 2002

Related U.S. Application Data
(62) Division of application No. 09/383,372, filed on Aug. 26, 1999.

(51) Int. Cl. A61K 39/395

(52) U.S. Cl. 424/145.1

(57) ABSTRACT

The use of endothelin inhibitors for the preparation of drugs for treatment or prevention of fibrotic disorders.

### DESCRIPTION

The invention relates to the use of endothelin inhibitors for the preparation of drugs for treatment or prevention of fibrotic disorders.

Endothelins are usually found at very low levels in the circulation which do not cause systemic effects. In pathological conditions, however, there may be a dramatic upregulation of the local endothelin system, both regarding the biologically active endothelins, particularly endothelin-1, and the endothelin receptors (type A and type B receptors), This local endothelin-endothelin receptor system acts in a juxta-, auto- and paracrine way to initiate a local but not systemic cellular response. Its activation results in very potent local vasoconstriction which on a molar level is approx. 100 times as effective as that caused by angiotensin II or catecholamines (E. R. Leven (1995) The New Engl. I. Med 333, 356-363). Endothelin receptor antagonism is not expected to have significant systemic effects on cells and tissues that are not injured or triggered to release endothelin or to upregulate endothelin receptors. This is best illustrated by the lack of adverse effect or of hypotensive reaction when high doses of endothelin receptor antagonists are administered to healthy people (G. Sütsch, O. Bertel, W. Kiowski, 1997, Cardiorasc Drugs Ther. 10, 717-725). This predisposes specific endothelin receptor antagonists as drugs with few or no side effects that prevent pathological processes resulting from a locally activated endothelin-endothelin receptor system.

There is evidence that endothelins are trophic factors for vascular smooth muscle cells, particularly during vascular and pulmonary hypertension (H. Weber, M. L. Webb, R. Serafino, et al. (1994). Mol. Endocrinol. 8:148-157; s. Eddahibi, B. Raffestin, M. Clozel, et al. (1995). Am. J. Physiol. 268:H828-835.). It is held that in hypertension the main source of local endothelin is the activated vascular endothelial cell which releases endothelin to the abluminal site to stimulate the vascular smooth muscle cell which via upregulation of its endothelin receptors responds by enhanced contraction and proliferation. Smooth muscle cell proliferation then results in vascular hypertrophy and perpetuation of the hypertensive state.

Importantly, recent studies indicate that special nonvascular cells, the myofibroblasts or myofibroblast-like cells which are the effector cells of all kinds of fibrotic processes in the body are important target cells for endothelin actions. These cells derive from usually quiescent mesenchymal cells in many organs (D. Schuppan, J. D. Jia, G. Boigk, C. Oesterling (1997). In: Recent Advnaces in the Pathophysiology of gastro-intestinal and liver diseases (J. P. Galmihce, J. Cournay, eds). John Libbey Eurotext, Montrouge, pp. 243-258; D. Schuppan, D. Strobel, E. G. Halm (1998). Digestion 59; 385-390.). Examples are the stellate cells (synonymous with lipocytes, Ito cells) and portal fibroblasts in the liver, the subepithelial and lamina propria fibroblasts in the intestine, the stellate cells and the interstitial fibroblasts in the pancreas, the mesangial cells and the interstitial fibroblasts in the kidneys, the alveolar and interstitial fibroblasts in the lungs, the interstitial fibroblasts in the heart, or the subepidermal and dermal fibroblasts in the skin.

The activated fibroblasts, myofibroblasts and myofibroblast-like cells of many tissues in vitro and in vivo usually increase their expression of procollagen I, the major collagen of fibrotic tissues, and also respond with enhanced DNA synthesis and proliferation. An important role for the activated endothelin-endothelin receptor system has been shown for the kidneys (B. Hocher, R. Zart, A. Schwarz, et al. (1998). J. Am Soc. Nephtrol. 9:1169-1177 H. Karem, D. Heudes, P. Bruneval, et al. (1996). Hypertension 28:379-385; B. Hocher, A. Lun, F. Priem, et al. (1998). J. Cardiovasc. Pharmacol. 31 (Suppl. 1):S492-495; B. Hocher, P. Rohmeiss, C. Thone-Reineke, et al. (1998). J. Cardiovasc. Pharmacol. 31 (Suppl. 1); S554-556.), the lungs (M. Uguccioni, L. Pulsatelli, B. Grigolo, et al. (1995). J. Clin. Pathol. 48:330-334; S. H. Park, D. Saleh, A. Giaid, R. P. Michel (1997). Am. J. Res. Crit. Care Med. 156:600-608; D. J. Abraham, R. Vancheeswaran, M. R. Dashwood, et al. (1997). Am. J. Pathol 151:831-841; R. K. Coker, G. J. Laurent (1998). Eur. Resp. J.11:1218 -1221), the heart (H. Karam, D. Heudes, P. Hess, et al. (1996). Cardiovasc. Res. 31:287-295; R. D. Forbes, P. Cernacek, s. Zheng, et al. (1996). Transplantation 61; 701-797, T. Suzuki, a. Tsuruda, S. Katoh, et al (1997). J. Mol. Cardiol. 29:1087-2093; P. Mulder, V. Richard, G. Derumeaux, et al. (1997). Circulation 96:1976-1982; M. Harada, H. Itoh, O. Nakagawa, et al. (1997). Circulation 96:3737-3744; H. Ju, s. Zhao, P. S. Tappia, et al. (1998). Circulation 97:892-899.), the skin in systemic sclerosis (F. M. Wigley. (1996). Curr. Opin. Rheumatol. 8:561-568; P. R. Ames, S. Lupoli, J. Alves, et al. (1997). Br. J. Rheumatol 36:1045-1050.), the pancreas (Y. Kakugawa, S. Paraskevas, P. Metrakos, et al. (1996).

Pancreas 13:89-95.) and the liver (M. Pinzani, s. Milani, R. De Franco, et al. (1996). Gastroenterology 110:534-548; D. C. Rockey, J. J. Chung (1996). J. Clin. Invest. 98:1381-1388; L. Racine-samson, D. C. Rockey, D. M. Bissell. (1997). J. Biol. Chem. 272:30911-30917; D. C. Rockey, L. Fouassier, J. J. Chung, et al. (1998). Hepatology 27:472-480.). The profibrogenic effect of endothelin is mediated by the A-receptor, whereas the B-receptor or B-may rather mediate antifibrogenic effects such as inhibition of myofibroblast proliferation and collagen synthesis, as was demonstrated for liver myofibroblasts (a. Mallat, L. Fouassier, A. M. Preeaux, et al. (1995). J. Clin. Invest. 96:42-29.).

Cho et al. (Z. Gastroenterologie 1998, 36, 769; Hepatology Vol28, No.4. Pt.2, 1998) disclose the effects of an endothelin receptor antagonist in rats with secondary biliary cirrhosis. They find that a specific blockade of the ETA receptor can reduce the collagen accumulation in biliary fibrosis, however it is accompanied by an increase in mortality.

Poo et al. Gastroenterology 116, 161-167 (1999)) describe that cirrhotic rats treated with an endothelin receptor antagonist showed a higher hydroxyproline content and procollagen mRNA expression than rats treated with placebo.

The object of the invention is to provide useful drugs for the prevention and therapy of fibrotic disorders in organs and tissues as lung, liver, skin, pancreas, kidney, heart, especially for the prophylaxis and treatment of fibrosis and cirrhosis of the liver.

This object is achieved by the use of endothelin inhibitors for the preparation of drugs for treatment or prevention of fibrotic disorders.

Endothelin inhibitors mean compounds which inhibit the effects of endothelin in a mammalian organism. This can be achieved by inhibiting the gene expression of endothelin for example by anti-sense technology or by compounds which inhibit specifically the transcription or translation of the endothelin gene or gene transcripts. Another way of inhibiting endothelin are compounds which bind specifically to endothelin and interrupt the communication of endothelin with its physiological partners. Such compounds are for example endothelin specific antibodies or fragments thereof or endothelin receptors or fragments thereof or low molecular weight compounds with a high affinity to endothelin.

Another class of endothelin inhibitors are compounds which inhibit the maturation of the active endothelin for example by inhibiting an endothelin converting enzyme.

A further class of endothelin inhibitors are compounds which bind to the endothelin specific receptors, so called endothelin receptor antagonists. These can be compounds which specifically bind to one class of the receptor without interfering with the other class of receptor, for example class A specific receptor antagonists (ETA receptor antagonists) or compounds which bind to both types of receptor ETA and ETB with similar affinity (mixed type ET receptor antagonists).

For the present invention ETA receptor antagonists are the preferred endothelin inhibitors, especially those which bind to the human ET receptor with an affinity constant Ki of 50 nMol/l or less, especially preferred 10 nMol/l or less.

Among the ETA receptor antagonists such compounds are preferred which bind with a selectivity factor of more than 50, preferred of more than 150, especially preferred of more than 250 to the ETA receptor. The selectivity factor is defined as Ki of a compound with respect to ETB receptor divided through Ki of the compound with respect to the ETA receptor.

Specifically preferred endothelin inhibitors are low molecular weight compounds disclosed in WO Ser. No. 96/11914, WO Ser. No. 97/38981 and WO Ser. No. 98/09953, especially those compounds which are listed individually in the tables.

### EXAMPLES OF SUITABLE ET-INHIBITORS INCLUDE

TBC-11251;

N-(4-Chloro-3-methylisoxazol-5-yl)-2-(2-(6-methyl-3,4-methylen-dioxy-1-yl)acetyl)thiophen-3-sulfonamide;

SE-209670;

(1S,2R,3S) 1-(3,4-methylendioxyphenyl)-3-(2-(carboxymethoxy)-4-methoxyphenyl)-5(prop-1-yloxy)indan-2-carboxylic acid;

Bosentan;

4-tert-Butyl-N-(6-(2-hydroxyethoxy)-5-(2-methoxyphenoxy)(2,2′-bipyrimidin)-4-yl)benzenesulfonamide;

PD-156707;

2-(3,4-Methylendioxyphenyl)-4-(4-methoxyphenyl)-4-oxo-3-(3,4,5-trimethoxybenzyl)-but-2-en acid sodium salt;

L-749329

4-(2-(4-isopropylphenylsulfonamido)-1-(3,4-methylenedioxyphe-nyl)-2-oxoethoxy)-3-propylbenzoic acid;

L-754142;

4-(2-(4-Isopropylphenyisulfonamido)-1-(3,4-methylandioxyphenyl)-2-oxoethoxy)-3-propylbenzoic acid di potassium salt;

SB-217242;

(1S,2R,3S) 1-(3,4-Methylendioxyphenyl)-3-(2-(2-hydroxyethoxy)-4-methoxyphenyl)-5(prop-1-yloxy)indan-2-carboxylic acid;

A-127722;

trans-trans-2-(4-methoxyphenyl)-4-(3,4-methylendioxyphenyl)-1-(2-(N,N-dibutylamino)-2-oxoethyl)-pyrrolidine-3-carboxylic acid;

ABT-627;

[2S-(2α, 3β,4α)]-2-(4-methoxyphenyl)-4-(3,4-methylendioxyphenyl)-1-(2-(N,N-dibutylamino)-2-oxoethyl)-pyrrolidine-3-carboxylic acid;

EMD-94246;

N-(2,1,3-Benzothiadiazol-5-yl)-5-(dimethylamino)naphthalin-1-sulfonamide potassium salt

ZD-1611;

3-(4-(3-(N-(3-Methoxy-5-methylpyrazin-2-yl)sulfamoyl)pyridin-2-yl)phenyl)-2,2-dimethylpropionic acid;

K-8794;

N-(2,6-dimethylphenyl)-3-(6-(4-t-butylphenylsulfonylamino)-5-(2-methoxyphenoxy) -2- (2-pyrimidinyl) -4-pyrimidinyloxy) propion-amide;

A-132086;

(2α, 3β,4α)-2-(3-Fluor-4-methoxyphenyl)-4-(3,4-methylen-dioxy-phenyl)-1-(2-(pentylsulfonyl)propylamino)ethyl-pyrrolidine-3-carboxylic acid;

PD-163070; PD-166557;

Ro-611790;

BMS-193884; BMS-207940;

SE-209598;

A-206377;

EMD-122801;

AC-61-0612;

T-0201;

J-104132

and compounds of the general formula I:

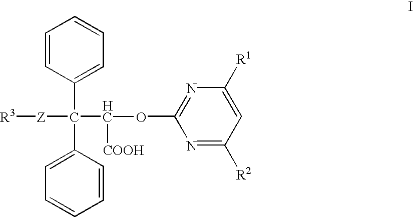

wherein R¹, R², R³, Z are:

R¹

C₁-C₄-Alkyl, C₁-C₄-Alkoxy;

R²

C₁-C₄-Alkyl, C₁-C₄-Alkoxy;

R³

C₁-C₈-Alkyl which may carry a phenyl which may carry up to 2 identical or different C₁-C₄-Alkoxy radicals;

Z

Oxygen or a single bond.

Preferred are compounds, wherein R¹, R², R³ and Z are:

R¹

C₁-C₂-Alkyl, C₁-C₂-Alkoxy;

R²

C₁-C₂-Alkyl, C₁-C₂-Alkoxy;

R³

C₁-C₂-Alkyl which may carry a phenyl which may carry up to 2 identical or different Cₗ-C₂-Alkoxy radicals;

Z

Oxygen or a single bond.

In therapeutic use, endothelin inhibitors may be administered by any route by which drugs are conventionally administered Such routes of administration include intraperitoneal, intravenous, intramuscular, subcutaneous, intrathecal, intraventricular, as well as oral. The administration route depends also on the nature of the endothelin inhibitor. For ET receptor antagonists the oral administration is the preferred one.

The dosage and length of treatment with endothelin inhibitors depends on the disease state being treated. The duration of treatment may be several weeks or longer and may, as a chronic therapeutic measurement or as a prophylactic treatment, last over the entire lifetime of the patient. The endothelin inhibitors are administered in a therapeutically effective amount; a typical human dosage of a endothelin inhibitor ranging from about 0.01 mg/kg of body weight to about 10 mg/kg, in single or repeated doses. The dosage will vary depending on the type of endothelin inhibitor used and its relative potency and pharmaco-kinetic properties.

As with fibrotic disorders, especially of the liver, the patient's capacity to metabolize drugs may be negatively impaired a dosage lower than the one applied in other endothelin mediated diseases may be useful for treatment of fibrotic disorders.

A lower dosage could be one which is 10 to 50% of the dosage used in other diseases with involvement of endothelin like hypertension or congestive heart failure.

Dosage and length of treatment are readily determinable by the skilled practitioner based on the condition and stage of disease.

The effective compounds can be administered in solid or liquid form in the conventional pharmaceutical administration forms, e.g. as tablets, suppositories, solutions, ointments, creams or sprays. These are prepared in a customary manner.

The active compounds can in this case be processed with the customary pharmaceutical auxiliaries such as tablet binders, fillers, preservatives, tablet disintegrants, flow-regulating agents, plasticizers, wetting agents, dispersants, emulsifiers, solvents, release-delaying agents, antioxidants and/or propellants (cf. H. Sucker et al.: Pharmazeutische Technologie [Pharmaceutical Technology], Thieme-Verlag, Stuttgart, 1991). The application forms thus obtained normally contain the active compound in an amount from 0.1 to 90% by weight

### METHODS

### Animal Experimentation

Female adult wistar rats, average weight 206±19 g, underwent the following microsurgical procedure under an operating microscope (OPMI 6-2, Zeiss, Germany) (B. Gerling, M. Becker, J. Waldschmidt, D. Schuppan (1996). J. Hepatol. 25:79-84; G. Boigk, L. Stroedter, H. Herbst H, et al. (1997). Hepatology 26:643-639.): 1. midline abdominal incision following anaesthesia with 100 mg/kg ketamine-hydroochloride (Ketanest®, Parke-Davis, Germany) and 10 mg/kg 5,6-dihydro-2-(2,6-xylidino)-4H-1,3-thiazine-hydrochloride (Rompun®, Bayer, Germany); 2. dissection of the common bile duct, insertion of a teflon catheter (Abbocath -T 26 G, venisystems, U.S.A.) and placement of a distal complete and a proximal incomplete ligature with 5-0 silk (Permahand , Ethicon, Germany); 3. retrograde injection of sodium-amidotrizoate (Ethibloc®, Ethicon Germany) at a dose of 0.02 ml/100 g body weight; 4. removal of the catheter, closure of the proximal ligature, scission of the bile duct between the ligatures and wound closure. After bile duct occlusion (BDO), animals received normal chow (Altromin®, Lage, Germany) and were allowed free access to water. The specific endothelin A receptor antagonist LU 135252 (LU, Knoll AG, Ludwigshafen, Germany) which had been mixed with the chow did not alter food consumption by the animals. The following therapeutic groups were formed: 1. BDO and LU at 80 mg/kg/d for 6 weeks (BDO 80/1-6:n=20); 2. BDO and LU at 80 mg/kg/d from week 4-6 of BDO (BDO 80/4-6:n=20); 3. BDO and LU at 10 mg/kg/d (sham 80/ 1-6:n=10), rats without medication (control: n=10) and bile duct occluded animals without treatment for 6 weeks (BDO: n=20) served as controls.

Early mortality (within 1 h to 3 days) in rats with BDO was due to local infection and amounted to 9%. Since in this model significant fibrosis is evident only after two weeks of BDO, these animals did not have to be considered for statistical analysis. After 6 weeks rats were sacrificed under ketanest(rompun-anaesthesia by puncture of the right ventricle and exsanguination. Heart, liver, spleen and kidneys were weighed, and 1-2 g pieces of the left and the right liver lobes fixed in 4% formalin for histology and hydroxyproline (HYP) determinations.

### Histological Methods

For each liver 1 μm paraffin sections of the right and the left lobe were stained with hematoxylin/eosin, trichrome (Masson-Goldner) and silver impregnation (Gomori), and scores (see below of both lobes averaged. Inflammation and necrosis were graded according to the histological activity index of Knodell et al. (R. G. Knodell, K. G. Ishak, W. C. Black, et al. (1981). Hepatology 1:431-435.). Staging of fibrosis was modified from (G. Boigk, L. Stroedter, H. Herbst H, et al. (1997). Hepatology 26:643-649.) as follows: stage 0; normal; stage I: portal fiels marginally expanded by bile ductules; stage II: expanded portal fields which occasionally contract each other; stage III: markedly expanded portal fields, all of which broadly contact each other, separating hepatocellular islands; stage IV: the liver is entirely filled with fibrotic biliary proliferations that entrap few residual hepatocytes (severe cirrhosis). Scores of the right and left lobes were averaged to give single numerical values for each liver.

### Analytical Methods

Hydroxyproline (HYP) was quantified in duplicates from 0.2 g of formalin-fixed liver as described (d. Schuppan, J. M. Dumont, K. Y. Kim, et al (1986). J. Hepatol. 3:27-37; C. Genovese; D. Rowe; B. Kream (1984). Biochemistry 23. 6210-6216.). Briefly, tissue was homogenized and hydrolyzed in 4 ml of 6 M HCl at 110° C. for 12 h, 50 μm of the filtered hydrolysates were evaporated under vacuum, the residue was dissolved in 1.2 ml of 50% isopropanol and incubated with 0.2 ml of 0.84% chloramine-T in 42 mM sodium acetate, 2.6 mM citric acid, 39.5% (v/v) isopropanol, pH 6.0 for 10 min at room temperature. 1 ml of a solution of p-dimethylaminobenzaldehyde (0.248 g dissolved in 11 ml of 60% perchloric acid) were added and 1.5 ml of this mixture were dissolved in 4 ml isopropanol and incubated at 50° C. for 90 min. Absorption was read at 558 nm and HYP concentrations were determined from a standard curve with 0 to 1.6 μg HYP (Merck, Germany). The hepatic HYP concentration was calculated using the formula

$$\frac{Absorption of sample}{0.26} \times 400 = μg HYP / g liver ,$$

and the total liver HYP content calculated by multiplication with the liver weight.

### Serum Measurements

Standard laboratory parameters were measured by our in-hospital clinical chemical department, using an automated analyser (BM/Hitachi 747). The aminoterminal propeptide of procollagen type III (PIIINP) was determined using a sequential saturation radioimmunoassay based on PIINP from rat, a monospecific rabbit antiserum to rat PIIINP and a goat antiserum to rabbit IgG, using a previously described procedure (B. Gerling, M. Becker, J. Waldschmidt, D. Schuppan (1996). J. Hepatol. 25:79-84; G. Boigk, L. Stroedter, H. Herbst H, et al. (1997). Hepatology 26:643-649; d. Schuppan, J. M. Dumont, K. Y. Kim, et al. (1986). J. Hepatol. 3:27-37.). Inter- and intra-assay coefficients of variation were 12% and 5% for a normal and 4% and 3% for a pathological serum sample, resp.

### RNAse Protection Assays

Plasmids encoded predescribed regions of the rat procollagen α1 (I) and glyceraldehyde dehydrogenase (GAPDH) genes, encompassing 230 bp (Bam HI/Rsal sites) and 102 bp (position 335-437), resp. (44, 45). The rat tissue inhibitor of metalloproteinase-1 (TIMP-1) probe (position 176-439) was prepared by RT-PCR according to the published sequence (J. P. Iredale, R. C. Benyon, M. J. P. Arthur (1996). Hepatology 24:176-184.), cloned into pZErO-1 (Invitrogen, San Diego, Calif., U.S.A.) and confirmed by restriction enzyme analysis. Preparation of riboprobes: The cDNA templates were linearized with appropriate restriction endonucleases. In vitro transcription was carried out in 10 μl of a mixture containing 0.5 μg of DNA, 50 μM each of ATP, CTP and GTP, 5 μM of UTP (50 μM UTP for GAPDH), 50 μCi of α-³²p-UTP (800 Ci/mmol, 10 mCi/ml; NEN Life Science, Boston, Mass., U.S.A.), 1 mM of dithiothreitol, 20 U of RNAsin (Promega, Madison, Wis., U.S.A.), 2 μl of 5 times transcription buffer and 5 U of bacteriophage T7 RNA polymerase (Promega). After incubation at 370° C. for 60 min, the transcription mixture was digested with 1 U of RNase free DNase (Promega) at 37° C. for 30 min. Riboprobes were purified by electrophoresis through a denaturing polyacrylamide gel and the radioactivity of eluted probes were measured by liquid scintillation counting. Multiple-probe ribonuclease protection assay (RPA): RPA was carried out with the RPA II kit (Ambion, Austin, Tex., USA) according to the manufacturer's instruction. In brief, 20 μg of total RNA, 30.000 cpm each of procollagen αl(I) and TIMP-1 probes, and 2.000 cpm of the GAPDH probe were hybridized in 20 μl hybridization buffer containing 80% formamide at 45° C. for 16 h. 200 μl of digestion buffer containing 40 U of RNase TI was added to the hybridization mixture and incubated at 37° C. for 1 h. The precipitated pellets were resuspended in 6 μl of loading buffer. After denaturation at 90° C. for 5 min, the mixture was run on a 5% polyacrylamide/8 M urea gel at 8 W for 90 min, followed by exposure to an x-ray film (Kodak, Rochester, N.Y., USA) at 70° C. for 16 h. Autoradiographic signals were analyzed with the public domain NTH Image Program. The procollagen α1(I) and TIMP-1 RNA signals were normalized to the signal of GAPDH RNA and expressed as relative abundance (arbitrary units). 10 liver samples each of negative controls, of untreated rats with BDO and of rats with BDO that received LU at 80 mg/kg/d were analyzed.

### Statistical Analysis

Data are presented as means±S.D, and as medians with 25./75. percentiles. statistical analysis was performed using the Mann-Whitney rank sum test. Differences in relative abundance of the RNAs were analyzed by the Kruskal-Wallis test and p<0.05 was regarded as statistically significant.

### Results and Discussion

A rat model of secondary biliardy liver fibrosis (bile duct obstruction by retrogarde injection of the sclerosant ethiblock, BDO) was developed that leads to a homogeneous and progressive accumulation of ECM, with a roughly tenfold increase of total hepatic collagen and the development of cirrhosis within 6 weeks. Importantly, development of fibrosis and cirrhosis in this model does not depend on necrosis and mononuclear cell infiltration which is found in other fibrosis models such as those induced by carbon tetrachloride, dimethylnitrosamine or galactosamine. Therefore, as shown previously, this model more truly reflects chronic liver disease in humans which are characterized by often little inflammation but ongoing fibrogenesisi Accordingly, several drugs that block radical formation or necrosis can prevent fibrosis and cirrhosis in the other rat models, but are ineffective in man as well as in rat secondary biliary fibrosis (D. Schuppan, J. D. Jia, C. Boigk, C. Oesterling (1997). In: Recent Advnaces in the Pathophysiology of gastro-intestinal and liver diseases (J. P. Galmihce, J. Gournay, eds). John Libbey Eurotext, Montrouge, pp. 243-258; D. Schuppan, D. Strobel, E. G. Halm (1998). Digestion 59:385-390; G. Boigk, L. Stroedter, H. Herbst H, et al. (1997). Hepatology 26:643-649.). Thus drugs that inhibit collagen accumulation in rat biliary liver fibrosis hold great promise for a similar effect in humans.

Rats with sham operation and rats with BDO were fed the oral endothelin A receptor antagonist LU 135252 (LU). LU at 80 mg/kg/d for 6 weeks did not alter any of the measured parameters (body and organ weights, liver acollagen, liver histology, clinical chemical values) when given to sham-operated rats (tables 1-3).

However, liver collagen (both as relative and total liver collagen content), which was increased 6- and 12-fold above normal, resp., in the untreated group with BDO, was reduced by 50-55% in rats on 80 mg LU/kg/d over the 6 weeks of BDO (p<0.001) (FIG. 1). Importantly, also the rats that received 80 mg LU/kg/d from week 4-6 after BDO, i.e., the group in which treatment was started when total liver collagen was already increase 4-5 fold above normal (at the end of the third week of BDO), exhibited a nearly 50% reduction of hepatic collagen (p<0.001). Since contrary to the sham-operated rats, LU at 80 mg/kg/d which represents a high dose, caused death dose-dependently due to rental tubular necrosis in 50% and 25% of the two treatment groups, resp., a second experiment with a lower dose (10 mg/kg/d) over 6 weeks of BDO was performed. Here, relative liver collagen was again reduced by 35% and no death or renal toxicity was observed (FIG. 1).

In rats with BDO neither nor the low dose groups demonstrated significant differences in the weights of kidneys, heart and lungs (not shown). However, LU reduced liver and spleen weights dose-dependently (table 1). The spleen weights are important, since their reduction correlates with a lowered portal pressure, the increase of which leads to often lethal complications of cirrhosis.

Apart form the significantly reduced liver collagen content, administration of LU also lead to an improved histological fibrosis score (table 2; correlation with relative liver collagen content. p<0.001, not shown). Necrossi and inflammation scored 0 except for 2 animals with a minimal score of 1 (not significant) Architectural distortion of the liver, which was semiquantitatively graded, was even more significantly reduced in the low-than in the high-dose group (not shown).

Clinical chemical serum parameters (aspartate aminotranferase, alanine aminotransferase, alkaline phosphatase, gamma glutamyltranspeptidase, total bilirubin, creatinine) did not differ between all groups with BDO (table 3). The almost normal transaminase values among rats with BDO again point to no or little inflammation and necrosis in this fibrosis model.

The serum aminoterminal propeptide of type III procollagen (PIIINP), a surrogate marker of liver fibrogenesis, was increased more than 10-fold in rats with BDO as compared to nonfibrotiic controls, and lowered dose-dependently by 20-50% in LU-treated animals (FIG. 1C). Again, as with histological scoring, liver hydroxyproline (representing collagen content) showed a good correlation with serum PIIINP values (p<0.00l, not shown). This is important, since PIIINP and other serum markers of hepatic ECM metabolism can also be determined in human sera, allowing a non-invasive monitoring of antifibrotic therapies (D. Schuppan, J. D. Jia, G. Boigk, C. Oesterling (1997). In: Recent Advnaces in the Pathophysiology of gastrointestinal and liver diseases (J. P. Galmihce, J. Gournay, eds). John Libbey Eurotext, Montrouge, pp. 243-258; D. Schuppan, D. Strobel, E. G. Halm (1998). Digestion 59:385-390; D. Schuppan, U. Stolzel, C. Oesterling, R. Somasundaram (1995). J. Hepatol. 22 (Suppl. 2):82-88.).

In order to further elucidate the antifibrotic mechanism of endothelin receptor antagonism in liver fibrosis, we used a multiprobe RNAse protection assay to determine the steady state levels of procollagen α1(I) and TIMP-1 RNA, which encode two of the most prominent profibrogenic proteins in fibrotic diseases. The high (80 mg/kg/d) dose of LU reduced these mRNA levels significantly by 35 and 55%, resp. (FIG. 2).

When liver sections were stained for desmin, an activation marker for rat hepatic stellate cells, the number of desmin-positive cells were significantly reduced in concert with the lowered collagen content in the LU-treated animals (data not shown), suggesting that, apart from suppression of procollagen and TIMP-1 expression, endothelin receptor antagonism also inhibits activation of mesenchymal cells to myofibroblasts and myofibroblast-like cells in vivo.

### REFERENCES

1. E. R. Leven (1995) The New Engl. I. Med 333, 356-363

2. G. Sütsch, O. Bertel, W. Kiowski, 1997, Cardiorasc. Drugs Ther. 10, 717-725

3. H. Weber, M. L. Webb, R. Serafino, et al. (1994). Mol. Endocrinol 8:148-157

4. s. Eddahibi, B. Raffestin, M. Clozel, et al. (1995). Am. J. Physiol. 268:H828-835.

5. D. Schuppan, J. D. Jia, G. Boigk, C. Oesterling (1997). In: Recent Advnaces in the Pathophysiology of gastro-intestinal and liver diseases (J. P. Galmihce, J. Gournay, eds). John Libbey Eurotext, Montrouge, pp. 243-258.

6. D. Schuppan, D. Strobel, E. G. Halm (1998). Digestion 59:385-390.

7. K. E. Dawes, A. D. Cambrey, J. S. Campa, et al. (1996) Int. J. Biochem. Cell Biol. 28:229-238.

8. B. Hocher, R. Zart, A. Schwarz, et al. (1998). J. Am Soc. Nephtrol. 9:1169-1177.

9. H. Karem, D. Heudes, P. Bruneval, et al. (1996). Hypertension 28:379-385.

10. B Hocher, A. Lun, F. Priem, et al. (1998). J. Cardiovasc. Pharmacol. 31 (Suppl. 1): S492-495.

11. B. Hocher, P. Rohmeiss, C. Thone-Reineke, et al. (1998). J. Cardiovasc. Pharmacol. 31 (Suppl. 1): S554-556.

12. M. Uguccioni, L. Pulsatelli, B. Crigolo, et al. (1995). J. Clin. Pathol. 48:330-334.

13. S. H. Park, D. Saleh, A. Giaid, R. P. Michel (1997). Am. J. Res. Crit. Care Med. 156:600-608.

14. D. J. Abraham, R. Vancheeswaran, M. R. Dashwood, et al. (1997). Am J. Pathol 151:S31-841

15. R. K. Coker, G. J. Laurent (1998). Eur. Resp. J.11:1218-1221.

16. H. Karam, D. Heudes, P. Hess, et al. (1996). Cardiovasc. Res. 31:287-295.

17. R. D. Forbes, P. Cernacek, s. Zheng, et al. (1996). Transplantation 61:701-797.

18. T. Suzuki, a. Tsuruda, S. Katoh, et al. (1997). J. Mol. Cardiol. 29:2087-2093.

19. P. Mulder, V. Richard, G. Derumeaux, et al. (1997). Circulation 96:1976-1982.

20. M. Harada, H. Itoh, O. Nakagawa, et al. (1997). Circulation 96:3737-3744.

21. H. Ju, s. Zhao, P. S. Tappia, et al. (1998). Circulation 97:892-899.

22. F. M. Wigley. (1996). Curr. Opin. Rheumatol. 8:561-568.

23. P. R. Ames, S. Lupoli, j. Alves, et al. (1997). Br. J. Rheumatol 36:1045-1050.

24. Y. Kakugawa, S. Paraskevas, P. Metrakos, et al. (1996). Pancreas 13:89-95.

25. M. Pinzani, s. Milani, R. De Franco, et al. (1996). Gastroenterology 110:534-548.

26. D. C. Rockey, J. J. Chung (1996). J. Clin. Invest. 98:1381-1388.

27. L. Racine-samson, D. C. Rockey, D. M. Bissell. (1997). J. Biol. Chem. 272:30911-30917.

28. D. C. Rockey, L. Fouassier, J. J. Chung, et al. (1998). Hepatology 27:472-480.

29. a. Mallat, L. Fouassier, A. M. Preeaux, et al. (1995). J. Clin. Invest. 96:42-29.

30. D. C. Rockey, R. A. Weisinger. (1996). Hepatology 24:233-240.

31. N. Kawada, K. Harada, K. Ikeda, K. Kaneda (1996). Cell Tissue Res. 286:477-486.

32. D. C Rockey (1997). Hepatology 25:2-5.

33. D. C. Mayer, L. A. Leinwand (1998). J. Cell Biol. 139:1477-1484.

34. S. L. Friedman (1993). N. Engl. J. Med. 328:1826-1835.

35. a. M. Gressner (1996). Kidney Int. /Suppl.) 54:839-845.

36. F. Grinnell (1994). J. Cell. Biol. 124:401-404.

37. e. J. Kovacs, L. A. DiPietro (1994). FASEB J. 8:854-861.

38. R. Ross (1993). Nature 362:801-809.

39. J. Floege, E. Eng. B. a. Young, R. J. Johnson (1993). Kidney Int. (Suppl.) 39:S47-S54.

40. B. Gerling, M. Becker, J. Waldschmidt, D. Schuppan (1996). J. Repatol. 25:79-84.

41. G. Boigk, L. Stroedter, H. Herbst H, et al. (1997). Hepatology 26:643-649.

42. R. G. Knodell, K. G. Ishak, W. C. Black, et al. (1981). Hepatology 1:431-435.

43. d. Schuppan, J. M. Dumont, K. Y. Kim, et al. (1986). J. Hepatol. 3:27-37.

44. C. Genovese; D. Rowe; B. Kream (1984). Biochemistry 23:6210-6216.

45. J. Y. Tso, x. H. Sun, T. H. Kao, et al. (1985). Nucl. Ac. Res. 13:2485-2492.

46. J. P. Iredale, R. C. Benyon, M. J. P. Arthur (1996). Hepatology 24:176-184.

47. D. Schuppan, U. Stolzel, C. Oesterling, R. Somasundaram (1995). J. Hepatol. 22 (Suppl. 2):82-88.

TABLE 1
Body, liver and spleen weights of rats

| | Control | Sham/LU | BDO | BDO/LU 80/1-6 | BDO/LU 80/4-6 |
|---|---|---|---|---|---|
| Groups | (n = 10) | (n = 10) | (n = 20) | (n = 10) | (n = 14) |
| B.W. (g) | 297.3 ± 18.15 | 305.8 ± 23.40 | 289.5 ± 22.37 | 293.0 ± 32.89 | 259.1 ± 30.2* |
| Liver W. (g) | 10.58 ± 1.78 | 11.98 ± 1.95 | 28.23 ± 3.09 | 24.73 ± 4.47* | 23.01 ± 5.33* |
| Spleen W. (g) | 0.68 ± 0.11 | 0.73 ± 0.08 | 2.25 ± 0.70 | 1.78 ± 0.47* | 1.62 ± 0.46 |

Control, normal diet for 6 weeks; Sham/LU; sham operation with LU 135242 (LU) at 80 mg/kg/d for 6 weeks; BDO, bile duct occlusion; BDO/LU 80/4-6, treated with LU at 80 mg/kg/d from week 4-6;
*significantly different from BDO alone (p < 0.05); all BDO significantly different from Control and Sham/LU (p < 0.001).

TABLE 2
Clinical chemical parameters

| | Control | Sham/LU | BDO | BDO/LU 80/1-6 | BDO/LU 80/4-6 | BDO/LU 80/1-6 |
|---|---|---|---|---|---|---|
| | (n = 10) | (n = 10) | (n = 20) | (n = 10) | (n = 14) | (n = 20) |
| ALT (U/L) | 31.9 ± 9.8 | 21.7 ± 3.3 | 26.3 ± 9.2 | 23.6 ± 10.8 | 40.2 ± 52 | 22 ± 8.4 |
| AST (U/L) | 54.8 ± 20.8 | 36.4 ± 7.9 | 179.4 ± 65* | 160.9 ± 78.5* | 229 ± 78.5*§# | 198 ± 56.7 |
| γGT (U/L) | 0 | 0.3 ± 0.5 | 16.6 ± 10.5* | 32.7 ± 24.4*§ | 28.1 ± 26.4* | 15 ± 6.4*# |
| ALP (U/L) | 105.5 ± 38.3 | 85.3 ± 17.9 | 325.8 ± 121.7* | 230.7 ± 55.7*§ | 253.1 ± 75.8*§ | 256.1 ± 49.6*§ |
| Bili | 1.3 ± 1.0 | 1.6 ± 0.5 | 193.8 ± 63.1* | 171.3 ± 49.5* | 209.9 ± 48.9* | 189.5 ± 43.2 |
| (μmol/L) | | | | | | |

Control, normal diet for 6 weeks; Sham/LU; sham operation with LU 135242 (LU) at 80 mg/kg/d for 6 weeks; BDO, bile duct occlusion; BDO/LU 80/4-6, treated with LU at 80 mg/kg/d from week 4-6; BDO/LU80/1-6, treated with LU at 80 mg/kg/d from week 1-6; BDO/LU10/1-6, treated with LU at 10 mg/kg/d from week 1-6. Values are means ± SD.
ALT, serum alanine aminotransferase; AST, aspartate aminotransferase; ALP, alkaline phosphatase; γ-GT, γ-glutamytranspeptidase; Bili, total bilirubin.
*significantly different from Control and Sham/LU (p < 0.001), § from BDO without treatment (p < 0.05), and # from LU80/1-6 (p < 0.05)

TABLE 3
Histological assessment of fibrosis

| Group | Control | Sham | BDO | BDO/LU 80/1-6 | BDO/LU 80/4-6 | BDO/LU 80/1-6 |
|---|---|---|---|---|---|---|
| Stage 0 | 10 | 10 | — | — | — | — |
| Stage I | — | — | — | 2 | 4 | 8 |
| Stage II | — | — | 3 | 4 | 5 | 5 |
| Stage III | — | — | 12 | 4 | 3 | 4 |
| Stage IV | — | — | 5 | | 2 | 3 |

Application of a semiquantitative scoring system. Cumulative scores of each individual LU-treated group is significantly better than the BDO alone group (p < 0.05).

### LEGENDS TO THE FIGURES

FIG. 1: Reduction of liver collagen and serum PIIINP by blocking the endothelin A receptor in biliary fibrotic rats

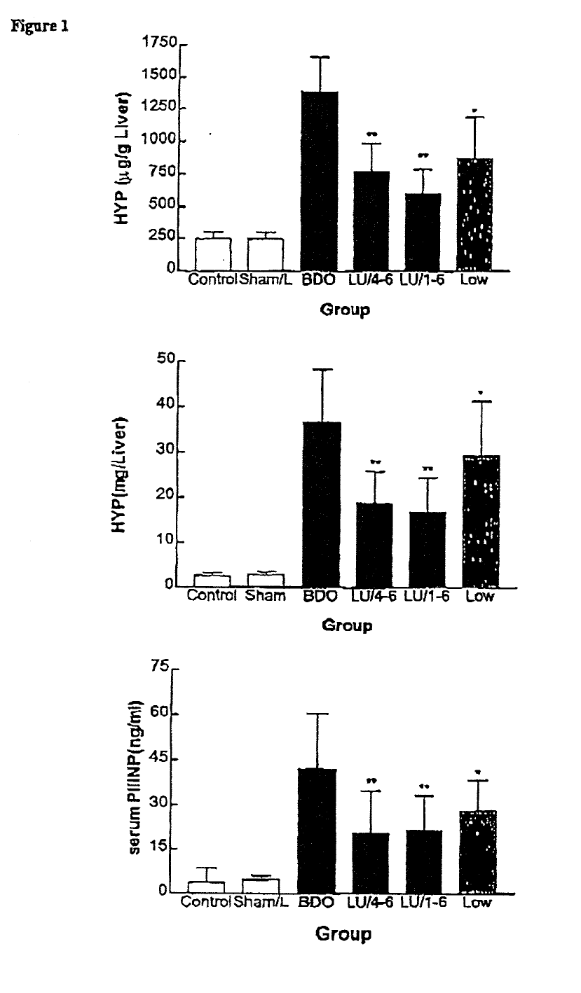

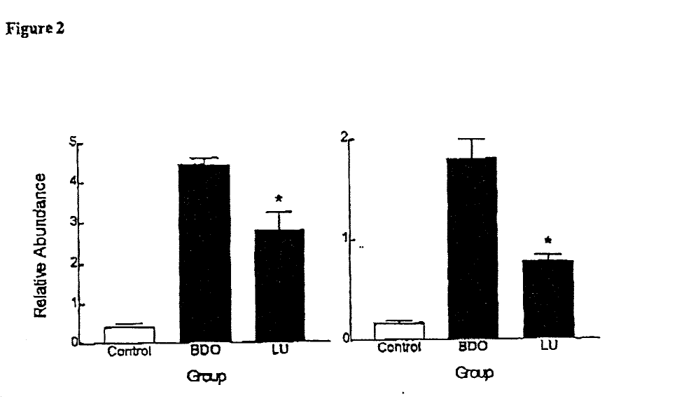

A: relative hepatic collagen content; B: total hepatic collagen content; C: PIIINP, serum aminoterminal propeptide of procollagen type III.

Hyp, hydroxyproline, as a measure of liver collagen. Control, normal diet for 6 weeks; Sham/LU; sham operation with LU 135242 (LU) at 80 mg/kg/d for 6 weeks; BDO, bile duct occlusion; BDO/LUSO/4-6, treated with LU at 80 mg/kg/d from week 4-6, BDO/LU80/1-6, treated with LU at 80 mg/kg/d from week 1-6; BDO/LUIO/1-6, treated with LU at 10 mg/kg/d from week 1-6. Values are means±SD. ALT, serum alanine aminotransferase; AST, aspartate aminotransferase; ALP, alkaline phosphatase; γ-GT, γ-glutamytran-speptidase; Bili, total bilirubin.

Significantly different from BDO alone (**p<0.001; *p<0.05).

FIG. 2: Reduction of procollagen a1(I) and TIMP-1 mRNA levels in biliary fibrostic rats treated with LU for 6 weeks.

Determination of RNA levels relative to GAPDH by multiprobe RNAse protection assay from untreated controls, rats with BDO and nor treatment and rats with BDO and LU at 80 mg/kg/d for 6 weeks (10 samples of each group).

Significantly different from BDO alone (*p<0.05).

1. The use of endothelin inhibitors for the preparation of drugs for treatment or prevention of fibrotic disorders.

2. The use according to claim 1 where the disorder is a fibrotic disorder of the liver.

3. The use according to claim 2 where the disorder is fibrosis or cirrhosis.

4. Pharmaceutical composition for prophylaxis or treatment of fibrotic disorders comprising as effective compound an endothelin inhibitor.

5. Pharmaceutical compositions according to claim 4 where the endothelin inhibitor is a endothelin receptor antagonist.

6. Pharmaceutical compositions according to claim 4 where the endothelin inhibitor is a specific ETA receptor antagonist.
